(19) United States
(12) Patent Application Publication
(10) Pub. No.: US 2006/0078600 A1
(43) Pub. Date: Apr. 13, 2006

(54) Transdermal therapeutic system suitable for heat application for promoting the permeation of active substances, and the use thereof

(75) Inventor: Walter Muller, Andernach (DE)

Correspondence Address: William F Lawrence;Frommer Lawrence & Haug, 745 Fifth Avenue, New York, NY, 10151, US

(73) Assignee: LTS Lohmann Therapie-Systeme AG, Andernach (DE)

(21) Appl. No.: 10/544,565

(22) PCT Filed: Feb. 3, 2004
(86) PCT No.: PCT/EP04/00944

§ 371 Date: Aug. 5, 2005

(30) Foreign Application Priority Data
Feb. 7, 2003 (DE) 103 04 989.4
Jan. 22, 2004 (DE) 10 2004 003 224.6

(51) Int. Cl. A61K 9/70; A61F 13/00

(52) U.S. Cl. 424/449; 604/20

(57) ABSTRACT

The invention relates to a transdermal therapeutic system (TTS) comprising at least one pharmacological active substance, which is suitable for heating up to a temperature above skin temperature, thereby promoting the permeation rate of the active substance. The transdermal therapeutic system comprises at least one layer containing the active substance and one peelable backing layer, and is characterize in that the layer containing the active substance mainly consists of a polysiloxane in which the active substance is partially dissolved.

Transdermal therapeutic systems (TTS) have now become an established pharmaceutical form which is also readily accepted by patients because of its therapeutic advantages. However, owing to the barrier effect of the human skin, transdermal administration of active substances having a topical and systemic effect is restricted to substances which are able because of their efficacy even at low doses and their physicochemical properties to permeate through the skin on an acceptable area in pharmacologically sufficient quantity.

There have been and there are therefore numerous efforts to influence the barrier effect of the skin and thus extend the range of active substances suitable for transdermal therapy. A first approach to achieving this aim was the use of specific substances, called permeation enhancers, which likewise diffuse into the skin and influence the structure of the stratum corneum, the main barrier to diffusion. Examples which may be mentioned are fatty alcohols, fatty acids and fatty acid esters, dimethyl sulfoxide, glycerol derivatives, polyoxyethylated fatty alcohols and fatty acids.

Besides this so-called chemical enhancement, also employed successfully have been physical methods such as electric current in so-called iontophoresis, ultrasound in so-called sonophoresis and heat.

The simplest physical means for increasing the permeability of the skin for chemical substances is the action of heat.

A transdermal system making use of this effect is described in U.S. Pat. No. 4,898,592. The transdermal system described therein optionally comprises a heat-conductive layer in order to utilize the heat of the body to increase the temperature at the site of application of the TTS.

Whereas this system operates purely passively, U.S. Pat. No. 4,230,105 describes a system which actively evolves heat after activation with water and thus increases the rate of permeation of active substances.

U.S. Pat. No. 5,919,479 describes a principle disclosing the application of controlled heat in combination with the application of local anesthetics. The heat is generated in this case by reaction of atmospheric oxygen with pyrophoric iron which is present on activated carbon in the presence of water.

U.S. Pat. No. 6,261,595 describes how heat-generating devices can be advantageously combined with conventional transdermal systems. It is pointed out that it is particularly advantageous for the transdermal systems to be configured in such a way that the heat-generating or releasing element can be mounted, removed or exchanged at any time without damaging the transdermal system. Easy replaceability or removability is important especially when the active substance delivery is to be adapted to the particular patient's requirements. This is particularly desirable for example when the active substance is an analgesic. Particularly with analgesics having many side effects, such as opiates, fentanyl and fentanyl-analogous substances, the patient should on the one hand in each case receive a sufficient quantity in order to be free of symptoms, but on the other hand no more than the quantity appropriate for the particular situation should be administered because of the side effects. It is possible by application of heat when pain occurs for the delivery of active substance by the system to be increased temporarily and thus adapted to the increased demand.

However, none of the above patents deals with the particular requirements for the properties of the matrix, the active substance-containing part of transdermal systems, on application of heat to increase the permeation rate.

It is an object of this invention therefore to develop formulations for the active substance-containing parts of a matrix system which are particularly suitable for increasing delivery of active substance through application of heat for the whole or a part of the administration time.

In the simplest case, matrix systems consist merely of an active substance-impermeable backing layer, of an active substance-containing and ideally self-adhesive matrix layer and of a protective layer to be removed before use. The active substance-containing layer normally consists besides the active substance for the most part of polymers based on polyacrylates, polyisobutylene, polyisoprene, styrene-polybutylene-styrene block copolymers, polysiloxanes, polyurethanes or hydrogels. Especially matrices based on polyisobutylene and block copolymers comprise additional tackifiers based on hydrocarbon resins and/or rosin derivatives. All matrices may comprise additional excipients which influence the solubility of the active substance or permeation enhancers which increase the rate of absorption from the system.

The control of active substance uptake in the case of matrix systems normally takes place less through the system but to a large extent through the skin itself. However, it is possible to provide the matrix on the skin side with a control membrane, and the membrane where appropriate with an adhesive layer for affixing the system to the skin in order thus to achieve better system control.

The adhesive layer on the skin side for affixing to the skin may likewise be loaded with active substance. This portion is then delivered without membrane control in order for example to saturate a possible skin depot for the active substance as quickly as possible.

The crucial parameter determining the delivery rate for the in vivo delivery of active substances with simple monolytic matrix systems is, besides the physicochemical properties of the active substance and the presence and efficacy of chemical permeation enhancers, the thermodynamic activity of the active substance. The maximum thermodynamic activity is reached in a stable system when the concentration of the active substance corresponds to the saturation concentration of the active substance in the matrix formulation. A further increase in the active substance concentration leads to no further increase in the thermodynamic activity if the proportion of the active substance which exceeds the saturation solubility is present as pure active substance, i.e. in the form of crystals or, in the case of low-melting active substances, as liquid phase, in the matrix. If the active substance is dissolved in the system in an amount exceeding the saturation concentration, the term used is supersaturated systems. Since the active substance is prone in such systems to recrystallize or form a separate phase during the storage time, such systems must be regarded as metastable or unstable.

The relationships described above show that there is a complex interaction between the current active substance concentration and the dissolving properties of the particular matrix formulation. These interactions must be taken into account especially with transdermal systems where the active substance delivery is increased through application of heat during the administration time or at least for part of the administration time.

The requirements to be met by such a system are as follows:
1. The thermodynamic activity of the active substance should be unchanged after application of external heat.
2. The thermodynamic activity of the active substance must return rapidly to the initial level after the application of heat.
3. The system must be designed so that, even when heat is applied and there is a temporary increase in the delivery rate associated therewith, the delivery of active substance at the desired level is ensured for the whole administration time, and the system is not exhausted prematurely.

The object is achieved according to the invention by a transdermal-therapeutic system as claimed in claim 1 of the present application.

The application therefore relates to a transdermal therapeutic system (TTS) suitable for administering at least one pharmacological active substance in a temperature range which is above skin temperature, comprising an active substance-impermeable backing layer, at least one active substance-containing matrix layer based on at least one polysiloxane, a detachable protective layer, and an internal and/or external heating element, characterized in that
a) the active substance-containing layer comprises, not taking the active substance into account, at least 80% by weight polysiloxane,
b) not more than 0.20% by weight of active substance is present in dissolved form, and
c) the active substance has a saturation solubility of not more than 5% by weight in the polysiloxane.

In one embodiment, the inventive TTS comprises an internal heating element, e.g. in the form of a matrix layer.

A preferred embodiment of the present invention is a TTS which is connected to an external heating element, called a heatpack, which can be removed or replaced without damaging the transdermal system. This element may, according to a further embodiment, also be present separate from the TTS but in a joint packaging unit.

The present invention further relates to the use of the above-defined TTS for the transdermal administration of active substances at temperatures above skin temperature, characterized in that, after removal of the protective layer, the matrix layer covered by the backing layer is applied to the skin and the matrix layer is heated for a certain time to a temperature above skin temperature either by an internal heating element or by connection of the TTS to an external heating element (called heatpack). In a preferred embodiment of the use, the matrix is heated to a temperature up to 50° C. inclusive.

The present invention further relates to a packaging unit in which the TTS and the external heating element (heatpack) are packed separate from one another.

Internal or external heating elements according to the invention may be those described for example in U.S. Pat. Nos. 5,919,479 and 6,261,595.

Polysiloxanes (more accurately: polyorganosiloxanes) can be obtained in the form of solutions or as solvent-free two-component systems. Variation in the organic part of the polysiloxanes results in a large number of different polysiloxanes, although only polydimethylsiloxanes have to date been applied for transdermal systems.

In two-component systems, the polysiloxane has a relatively low molecular weight and is fluid to viscous at room temperature. Only when the two components are mixed does crosslinking, leading to a usable product in each case, take place.

Transdermal systems are preferably produced by employing dissolved polydimethylsiloxanes of the following structural formula.

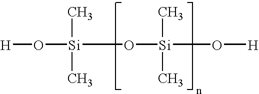

The additional use of monomethylsiloxane as monomer in this case achieves a certain three-dimensional, cohesion-improving crosslinking.

In further polysiloxanes of the invention it is possible for the methyl groups to be wholly or partly replaced by other alkyl radicals or phenyl radicals.

The molecular weight and the degree of crosslinking are important parameters which determine the cohesion and the adhesiveness of the polymer after removal of the solvent.

Because of the free OH groups, polysiloxanes of the above structural formula are prone in the presence of basic substances, e.g. amines, to condensation reactions which reduce the adhesiveness over the course of time. In a particular amine-resistant-form of these polymers, therefore, all free Si—OH groups, are converted into Si—O—C(CH₃)₃ groups.

In general, polysiloxanes are distinguished by good compatibility with skin, a low dissolving capacity for most active substances and an extremely high diffusion coefficient for the dissolved portion of the active substance. For the purposes of this invention, specifically the low dissolving capacity and the high diffusion coefficient are particularly important.

Because of the low dissolving capacity, most of the active substance is undissolved even with a small loading of active substance. It is possible in every case by suitable choice of the ratio of active substance to polysiloxane to ensure when the solubility of the active substance is not more than 5% by weight in the matrix that at least 80% of the active substance is in undissolved form. Since therefore only a small part of the amount of active substance to be delivered during the administration time is present in dissolved form in the matrix, subsequent dissolving of the active substance and its rapid diffusion to the delivery area is important in order to keep the concentration of the dissolved active substance in the matrix near to the saturation concentration and thus ensure a continuously high thermodynamic activity of the active substance over the administration period. Polysiloxanes also satisfy this condition in an ideal manner because of their high diffusibility.

On external application of heat, the TTS and thus the matrix are heated to temperatures of up to 50° C. It is to be assumed in this connection that the increase in temperature will always result in an increase in the saturation solubility of the active substance and an increase in the concentration of dissolved active substance. Since the thermodynamic activity of the active substance in the matrix and the diffusion coefficient of the active substance in the stratum corneum is increased thereby, the rate of delivery of the active substance from the TTS to the body increases.

Since on returning to normal conditions (i.e.: skin temperature) the amount of dissolved active substance initially remains unchanged, the matrix is supersaturated because the saturation solubility is now reduced again. Since this effect leads to a thermodynamic activity which is now likewise raised at skin temperature, and thus to an increased active substance flux through the skin, it is important for the supersaturation to be eliminated rapidly. Polysiloxanes as basic polymer for matrices satisfy this condition in an ideal manner because of their poor solubility for active substances since, owing to the low saturation solubility of active substances in such matrices, the concentration of the partly dissolved active substance returns within a short time to the saturation concentration due to the delivery of active substance to the body. A return to saturation solubility through recrystallization of the portion of active substance which is dissolved above the saturation solubility is only theoretically possible because active substances and other dissolved substances crystallize out only with difficulty or very slowly in polymers.

The facts described above can be illustrated by a model calculation on 2 typical TTS. This entails comparison of a TTS which represents the most unfavorable case of a TTS based on a silicone adhesive for the purposes of this invention with a thoroughly typical TTS based on polyacrylate adhesives.

Assumptions:

| | |
|---|---|
| TTS area: | 10 cm² |
| Active substance loading: | 20 mg/TTS |
| Saturation solubility in silicone system: | 5% (g/g) |
| Coating weight of silicone system: | 80 g/m² |
| Saturation solubility in polyacrylate | 20% |
| system: | |
| Coating weight of polyacrylate system: | 50 g/m² |
| Active substance delivery: | 50 μg/h |
| Temperature increase: | 32° C. → 40° C. |
| Increase in the saturation solubility at | 10% |
| 40° C.: | |

| | | | Polyacrylate | |
|---|---|---|---|---|
| | Silicone TTS | | TTS | |
| Weight of matrix | 16 | mg | 10 | mg |
| Active substance loading | 4 | mg | 4 | mg |
| of which dissolved at 32° C. | 0.8 | mg¹⁾ | 2 | mg²⁾ |
| of which dissolved at 40° C. | 0.88 | mg | 2.2 | mg |
| Time to return to saturation | 1.6 | hours | 4.0 | hours |
| solubility at 32° C.³⁾ | | | | |

¹⁾Saturation solubility 5% by weight; equivalent to 20% of the total amount of active substance
²⁾Saturation solubility 20% by weight, equivalent to 50% of the total amount of active substance
³⁾Calculation:

$$\frac{( \begin{matrix}mg of active substance dissolved at {40}^{°} C - \\ mg of active substance dissolved at {32}^{°} C\end{matrix} ) * 1000}{delivery rate [ μ g \text{/} h ]}$$

As somewhat of a simplification, it was assumed for this that the active substance delivery immediately after the end of the heat application is again 50 μg/h hour.

Qualitative, the differences in behavior of the two TTS are illustrated in the following drawing.

FIG. 1: Behavior of silicone TTS and polyacrylate TTS after application of heat to increase the permeation rate

In a TTS which already contains the active substance initially below the saturation concentration, subsequent dissolving of active substance is not possible when, as a result of the rise in temperature, the saturation solubility increases and the difference between actual concentration and saturation concentration becomes larger. The increase in the permeation rate is therefore less compared with saturated systems, with the increase in the diffusion coefficient in the stratum corneum making the essential contribution. When the permeation rate in such systems is increased by heating, the active substance concentration is reduced more quickly due to the increased delivery of active substance during the application of heat. This means that, on returning to normal conditions, the delivery of active substance is reduced as a function of the additionally delivered amount of active substance. Hence, such subsaturated systems are substantially useless in conjunction with applications of heat to increase permeation rates when a constant delivery of active substance over the administration period between the phases of application of heat is required. Only systems with a high active substance loading and a low delivery of active substance are suitable under this condition, because the active substance concentration and thus the delivery rate changes relatively little under these conditions during the administration time or through the increased delivery of the active substance during an application of heat.

However, this is achieved at the expense of a smaller utilization of active substance and such systems are uneconomic for costly active substances.

TTS for the purposes of this invention can be produced by suspending the active substance in the solution of the polysiloxane, adding all the other excipients and homogenizing the composition by stirring. After coating onto a suitable sheet, the solvents of the adhesive are evaporated off and the dried film is laminated with the backing layer of the TTS. The finished TTS are then punched out of this laminate. The complete procedure is described by way of Example 1 with fentanyl as base. The production of a TTS with an additional control membrane is described in Example 2.

Active substance which may be mentioned as suitable for the transdermal therapeutic system of the invention are the following:

analgesics such as, for example, fentanyl or a fentanyl-analogous substance, butorphanol, oxicodone, ketorolac, buprenorphine, morphine, tetracaine, lidocaine, bupivacaine, prilocalne and benzocaine;

antiparkinson medicaments such as, for example, biperiden, selegillin, pramipexol bronchospasmolytics such as, for example, salbutamol, tulobuterol

antiepileptics such as, for example, clonazepam, tiagabine

### EXAMPLE 1

### TTS with Fentanyl as Active Substance Without Control Membrane

3 g of fentanyl base are added to 138 g of a 70 percent solution of a silicone adhesive in n-heptane (BIO-PSA 4301, Dow Corning) and suspended in this solution by stirring. The suspension is then coated onto a suitable removable protective sheet (Scotchpak 1022, 3M) in a thickness resulting in a coating weight of 100 g/m² after removal of the solvent. The dried film is then laminated with a 23 μm-thick polyester sheet, the backing layer of the TTS, and the TTS is punched out of this laminate. The individual TTS are packaged in a bag made of a heat-sealable packaging material laminate.

### EXAMPLE 2

### TTS with Fentanyl as Active Substance with Control Membrane

a. Production of the Reservoir Layer

3 g of fentanyl base are added to 138 g of a 70 percent solution of a silicone adhesive in n-heptane (BIO-PSA 4301, Dow Corning) and suspended in this solution by stirring. The suspension is then coated onto a suitable removable protective sheet (Scotchpak 1022, 3M) in a thickness resulting in a coating weight of 100 g/m² after removal of the solvent. The dried film is then laminated with a 23 μm-thick polyester sheet, the backing layer of the TTS.

b. Production of the Skin-Adhesive Layer

The solution of a silicone adhesive with a solids content of 70% in n-heptane (BIO-PSA 4301, Dow Corning) is coated onto a suitable removable protective sheet (Scotchpak 1022, 3M) in a thickness resulting in a coating weight of 30 g/m² after removal of the solvent. The control membrane (50 μm-thick EVA film with a vinyl acetate content of 19%) is then laminated onto the dried film.

c. Production of the Complete Laminate

The removable protective sheet is peeled off the reservoir layer produced under a, and the skin-adhesive layer produced under b is laminated onto the membrane. The individual TTS are punched out of the complete laminate and packaged in a bag made of a heat-sealable packaging material laminate.

1. A transdermal therapeutic system (TTS) for administering at least one pharmacological active substance at a temperature which is above skin temperature, comprising at least one active substance-impermeable backing layer, at least one matrix layer which is based on at least one polysiloxane and contains at least one pharmacological active substance, a detachable protective layer, and an internal and/or external heating element, characterized in that
a) the active substance-containing layer comprises, not taking the active substance into account, at least 80% by weight of at least one polysiloxane,
b) not more than 20% by weight of active substance is present in dissolved form, and
c) the active substance has a saturation solubility of not more than 5% by weight in the polysiloxane.

2. The TTS as claimed in claim 1, characterized in that it includes an internal heating element.

3. The TTS as claimed in claim 1, characterized in that it includes an external heating element.

4. The TTS as claimed in claim 1, characterized in that it is suitable for administering an active substance at a temperature up to 50° C. inclusive.

5. The TTS as claimed in claim 1, characterized in that the polysiloxane is amine-resistant.

6. The TTS as claimed in claim 1, characterized in that the polysiloxane has self-adhesive properties.

7. The TTS as claimed in claim 1, characterized in that the polysiloxane is a polydimethylsiloxane.

8. The TTS as claimed in claim 1, characterized in that the active substance-containing layer is provided on the skin side with a control membrane, and the latter is optionally provided with an adhesive layer for affixing to the skin.

9. The TTS as claimed in claim 8, characterized in that the adhesive layer for affixing to the skin is likewise loaded with active substance.

10. The ITS as claimed in claim 1, characterized in that the active substance-containing layer comprise an excipient which reduces the barrier properties of the skin.

11. The TTS as claimed in claim 10, characterized in that the excipients which reduces the barrier properties of the skin is oleic acid.

12. The TTS as claimed in claim 1, characterized in that the active substance is an analgesic having topical or systemic activity.

13. The TTS as claimed in claim 12, characterized in that the active substance is fentanyl or a fentanyl-analogous substance, butorphanol, oxycodone, ketorolac, buprenorphine, morphine, tetracaine, lidocaine, bupivacaine, prilocalne or benzocaine.

14. The TTS as claimed in claim 1, characterized in that the active substance is an antiparkinson agent, a vaccine, a bronchospasmolytic, an antiepileptic or an anticraving agent.

15. The use of a TTS as claimed in claim 1 for transdermal administration of at least one pharmacological active substance at temperatures above skin temperature, characterized in that, after removal of the protective layer, the matrix layer covered by the backing layer is applied to the skin and the matrix layer is heated for a certain time to a temperature above skin temperature either by an internal heating element or by connection of the matrix layer of the TTS to an external heating element (heatpack).

16. The use of a TTS as claimed in claim 15, characterized in that the matrix layer is heated to a temperature up to 50° C. inclusive with the aid of an external heating element.

17. A packaging unit, characterized in that it comprises, packaged individually,
a) a TTS as claimed in claim 1, but without the internal and without the external heating element, and
b) one or more external heating elements (heatpacks).
